(19) United States
(12) Patent Application Publication
(10) Pub. No.: US 2016/0059001 A1
(43) Pub. Date: Mar. 3, 2016

(54) IMPLANTABLE MEDICAL LEAD AND METHOD OF MAKING SAME

(71) Applicant: PACESETTER, INC., Sylmar, CA (US)

(72) Inventors: Keith Victorine, Santa Clarita, CA (US); Sergey Safarevich, Whippany, NJ (US); Steven R. Conger, Agua Dulce, CA (US); Serdar Unal, Los Angeles, CA (US); Sean Matthew Desmond, Moorpark, CA (US)

(21) Appl. No.: 14/935,074

(22) Filed: Nov. 6, 2015

Related U.S. Application Data
(62) Division of application No. 13/840,389, filed on Mar. 15, 2013.

(51) Int. Cl. A61N 1/05; B23K 26/32; B23K 26/21

(52) U.S. Cl. CPC A61N 1/05; A61N 1/0563; B23K 26/21; B23K 26/32; B23K 2203/05

(57) ABSTRACT

An implantable medical lead may include a longitudinally extending body, an electrical conductor, an electrical component, and a weld. The longitudinally extending body includes a distal end and a proximal end. The electrical conductor extends through the body between the proximal end and the distal end. The electrical component is on the body and includes a sacrificial feature defined in a wall of the electrical component. The sacrificial feature includes a region that continues from the wall of the electrical component and a side that is isolated from the wall of the electrical component via a void defined in the wall of the electrical component. The weld is formed at least in part from at least a portion of the sacrificial feature. The weld operably couples the electrical component to the electrical conductor.

### CROSS REFERENCE TO RELATED APPLICATIONS

This application is a division of U.S. patent application Ser. No. 13/840,389, filed Mar. 15, 2013, and is related to U.S. patent application Ser. No. 13/840,642, filed Mar. 15, 2013, and is incorporated herein by reference in its entirety.

### FIELD OF THE INVENTION

The present invention relates to medical apparatus and methods. More specifically, the present invention relates to implantable medical leads and methods of manufacturing such leads.

### BACKGROUND OF THE INVENTION

Implantable pulse generators, such as pacemakers, defibrillators, implantable cardioverter defibrillators (“ICD”) and neurostimulators, provide electrotherapy via implantable medical leads to nerves, such as those nerves found in cardiac tissue, the spinal column, the brain, etc. Electrotherapy is provided in the form of electrical signals, which are generated in the pulse generator and travel via the lead's conductors to the electrotherapy treatment site.

Patients may benefit from electrotherapy treatments to be proposed in the future. However, current conventional lead manufacturing technology has generally limited the extent to which leads can be reduced in size and the elements or features that can be carried on leads.

There is a need in the art for a lead having a configuration that allows the lead to have a reduced size and which is capable of supporting elements or features in a variety of configurations. There is also a need in the art for a method of manufacturing such a lead and manufacturing methods that reduce the cost of such leads.

### BRIEF SUMMARY OF THE INVENTION

An implantable medical lead is disclosed herein. In one embodiment, the lead may include a longitudinally extending body, an electrical conductor, an electrical component, and a weld. The longitudinally extending body includes a distal end and a proximal end. The electrical conductor extends through the body between the proximal end and the distal end. The electrical component is on the body and includes a sacrificial feature defined in a wall of the electrical component. The sacrificial feature includes a region that continues from the wall of the electrical component and a side that is isolated from the wall of the electrical component via a void defined in the wall of the electrical component. The weld is formed at least in part from at least a portion of the sacrificial feature. The weld operably couples the electrical component to the electrical conductor.

In one embodiment of the lead, the electrical component includes a ring electrode or a defibrillation coil. In one embodiment of the lead, the electrical component includes a strain gage, a pressure sensor, a piezoelectric sensor, an integrated chip, an inductor, or a position tracking sensor. In one embodiment of the lead, the electrical component includes a header. A helical anchor extendable from within a distal tip of the lead for active fixation may be at least partly provided in a lumen of the header. In one embodiment of the lead, the electrical component includes a ring contact in a lead connector end.

In one embodiment, the lead further includes a crimp secured to the electrical conductor, and the weld is also formed at least in part from at least a portion of the crimp. The crimp may include a crimp-thru type crimp.

In one embodiment, the sacrificial feature includes a welding tab. The welding tab may be considered peninsular within the void defined in the wall of the electrical component.

In one embodiment of the lead, the wall of the electrical component includes a distal end edge and a proximal end edge, and the void is defined in the wall between, and spaced away from, the distal end edge and the proximal end edge. In another embodiment of the lead, the wall of the electrical component includes a distal end edge and a proximal end edge, and the void is defined in the wall at either the distal end edge or the proximal end edge.

In one embodiment of the lead, a longitudinal axis of the sacrificial feature extends along a longitudinal axis of the electrical conductor at the weld.

A method of assembling an implantable medical lead is also disclosed herein. In one embodiment, the method includes: supporting an electrical component on a lead body, the electrical component including a sacrificial feature defined in a wall of the electrical component, the sacrificial feature including a region that continues from the wall of the electrical component and a side that is isolated from the wall of the electrical component via a void defined in the wall of the electrical component; and welding at least a portion of the sacrificial feature, a resulting weld operably coupling the electrical component to an electrical conductor extending through the lead body.

In one embodiment of the method, the electrical component includes a ring electrode or a defibrillation coil. In one embodiment of the method, the electrical component includes a strain gage, a pressure sensor, a piezoelectric sensor, an integrated chip, an inductor, or a position tracking sensor. In one embodiment of the lead, the electrical component includes a header. A helical anchor extendable from within a distal tip of the lead for active fixation may be at least partly provided in a lumen of the header. In one embodiment of the lead, the electrical component includes a ring contact in a lead connector end.

In one embodiment, the method further includes securing a crimp to the electrical conductor, and also welding at least a portion of the crimp to form at least a part of the resulting weld. The crimp may be secured to the electrical conductor via a crimp-thru type crimping process.

In one embodiment of the method, the sacrificial feature includes a welding tab. Prior to welding the at least a portion of the sacrificial feature, the welding tab is peninsular within the void defined in the wall of the electrical component.

In one embodiment of the method, the wall of the electrical component includes a distal end edge and a proximal end edge, and the void is defined in the wall between, and spaced away from, the distal end edge and the proximal end edge. In another embodiment of the method, the wall of the electrical component includes a distal end edge and a proximal end edge, and the void is defined in the wall at either the distal end edge or the proximal end edge.

In one embodiment of the method, prior to welding the at least a portion of the sacrificial feature, a longitudinal axis of the sacrificial feature extends along a longitudinal axis of the electrical conductor at the weld.

In one embodiment of the method, prior to welding the at least a portion of the sacrificial feature, the sacrificial feature includes a peninsular shape within the void, the peninsular shape including a trapezoidal shape, a rounded rectangular shape, or a conical shape terminating in a circular free end.

An implantable medical lead is disclosed herein. In one embodiment, the lead may include a longitudinally extending body, a first and second electrical conductor, a first and second electrical component, and a first and second weld. The longitudinally extending body includes a distal end and a proximal end. The first and second electrical conductors extend through the body between the proximal end and the distal end. The first electrical component is positioned on the body relative to the second electrical component. The first and second electrical components each include a sacrificial feature defined in a wall. The sacrificial feature includes a region that continues from the wall and a side that is isolated from the wall via a void defined in the wall. The first weld is formed at least in part from at least a portion of the sacrificial feature of the first electrical component, and the second weld is formed at least in part from at least a portion of the sacrificial feature of the second electrical component. The first weld operably couples the first electrical component to the first electrical conductor, and the second weld operably coupled the second electrical component to the second electrical conductor.

In one embodiment of the lead, the first and second electrical components form a split ring electrode.

In one embodiment, the lead further includes a first crimp secured to the first electrical conductor and a second crimp secured to the second electrical conductor. The first weld is also formed at least in part from at least a portion of the first crimp, and the second weld is also formed at least in part from a portion of the second crimp. The first and second crimps may include a crimp-thru type crimp.

In one embodiment, the sacrificial features of the first and second electrical components each include a welding tab. The welding tab may be considered peninsular within the void defined in the wall of each of the first and second electrical components.

An implantable medical lead is disclosed herein. In one embodiment, the lead may include a longitudinally extending body, a structure, a mechanical component, and a weld. The longitudinally extending body includes a distal end and a proximal end. The structure is supported by the body. The mechanical component is on the body and includes a sacrificial feature defined in a wall of the mechanical component. The sacrificial feature includes a region that continues from the wall of the mechanical component and a side that is isolated from the wall of the mechanical component via a void defined in the wall of the mechanical component. The weld is formed at least in part from at least a portion of the sacrificial feature. The weld operably couples the mechanical component to the structure

In one embodiment, the mechanical structure is a header. In one embodiment, the mechanical component is an actuation member such as, for example, a pull cable.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable medical lead and a pulse generator for connection thereto.

FIG. 2 is an isometric view of the ring electrode employing a peninsular welding tab for welding to a crimp-thru crimp crimped to an electrical conductor extending through the lead body.

FIG. 2A is the same view of the ring electrode with the same peninsular welding tap configuration, except the welding tab is oriented differently.

FIG. 3 is the same view as FIG. 2, except of a ring electrode employing opposed multiple peninsular welding tabs.

FIG. 4 is the same view as FIG. 2, except of a ring electrode having an alternatively shaped peninsular welding tab.

FIG. 5 is the same view as FIG. 2 except of a ring electrode having another alternatively shaped peninsular welding tab located at an end of the ring electrode.

FIGS. 6-9 are successive plan views of the ring electrode being mounted on the lead body and electrically and mechanically coupled to the conductor.

FIG. 10 is a flow chart outlining the method assembly method depicted in FIGS. 6-9.

FIG. 11 is an isometric view of a split ring electrode employing a plurality of peninsular welding tabs.

FIG. 12 is a cross-sectional view of a split ring electrode employing a plurality of peninsular welding tabs, each for welding to a crimp-thru crimp crimped to one of a plurality of electrical conductors extending through the lead body.

FIG. 13 is a plan view of a plurality of split ring electrodes mounted on the lead body and electrically and mechanically coupled to a plurality of conductors.

FIG. 14A is a perspective cross-sectional view of a first location where a first electrical component of a first split ring electrode is electrically and mechanically coupled to an electrical conductor and a second location where a first electrical component of a second split ring electrode is electrically and mechanically coupled to an electrical conductor.

FIGS. 14B-14C are cross-sectional views of the first location and the second location of FIG. 14A, respectively.

FIG. 15 is an isometric view of a header employing a peninsular welding tab.

FIG. 16 is the same view as FIG. 15 except of a header having an alternatively shaped peninsular welding tab located at an end of the header.

FIG. 17 is a plan view of a lead connector end employing a peninsular welding tab.

FIGS. 18A-18B show an extended peninsular welding tab before and after welding, respectively.

### DETAILED DESCRIPTION

An implantable medical lead 10 is disclosed herein. In one embodiment, the implantable medical lead 10 includes a longitudinally extending body 50, an electrical conductor 100, and an electrical component 80, such as, for example, an electrode for sensing or pacing, a defibrillation coil, a strain gage, a pressure sensor, a piezoelectric sensor, an integrated chip, an inductor, a position tracking sensor, a header, etc. The body 50 includes a distal end 45 and a proximal end 40. The electrical conductor 100 extends through the body 50 between the proximal end 40 and the distal end 45 and includes a location 110 along its length wherein the electrical component 80 is electrically and mechanically coupled to the electrical conductor 100.

In one embodiment, the location 110 on the electrical conductor 100 may additionally include a thin-walled, crimp-thru crimp 120 crimped to the location 110 on the conductor 100 to electrically and mechanically couple the crimp 120 the electrical conductor 100. In other embodiments, the crimp 120 may be a tube 120 or other structure that is welded or otherwise mechanically and electrically coupled to the electrical conductor 100 at the location 110. In being so coupled to the electrical conductor 100 at the location 110, the crimp or tube 120 may extend about at least a portion of an outer circumference of the electrical conductor 100 at the location 110.

To facilitate the welding of the electrical component 80 to the electrical conductor 100 directly or via the intervening crimp or tube 120, the electrical component 80 includes an isolated, sacrificial welding tab 125. Employing welding tabs 125 as disclosed herein in the manufacture of leads 10 offers a number of benefits. First, a successful weld requires less energy when employing the welding tab 125 due to the concentration of the heat on the welding tab 125. Stated differently, by isolating the sacrificial welding tab 125, the heat generated from welding is concentrated in a localized area, thereby reducing the welding heat propagating into the lead body 50 and the underlying crimp 120. By concentrating the heat on the welding tab 125, a low energy weld may be performed. Second, employing welding tabs 125 facilitates crimp-thru technology, which reduces the overall size and cost of a lead 10. Third, employing welding tabs 125 facilitates the use of thinner walled crimps, which helps to reduces lead diameter. Fourth, less intimate contact between metal parts prior to welding is required for a consistent and reliable weld when employing welding tabs 125. Fifth, employing welding tabs 125 provides a controlled welding process due to consistent heat transfer in parts subjected to welding because there is a controlled heat sink region, thereby making the welding process and resulting weld more forgiving and less operator dependant. Finally, the welding tab 125 is also more conformal during welding as it allows for greater and more controlled flow of the molten metal between the electrode and crimp sleeve, thereby resulting in more consistent welds to thinner walled crimps and facilitating the downsizing in the diameter of lead bodies. As a result of these benefits, lead manufacturing costs are reduced, smaller diameter lead bodies are facilitated, electrical insulation jackets of electrical conductors 80 are not degraded or otherwise damaged by the welding, and welds are less likely to become contaminated and weak during the welding process.

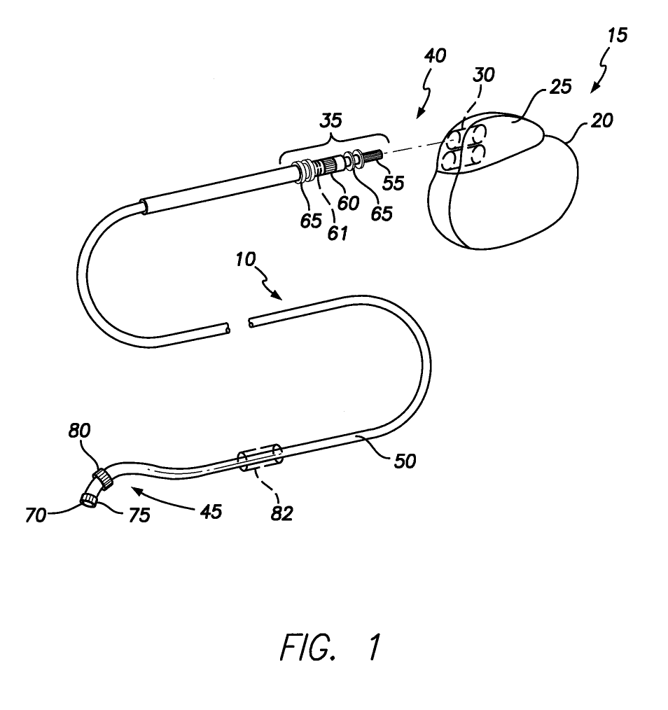

For a general discussion of an embodiment of a lead 10 employing the above-described tabbed welded connection, reference is made to FIG. 1, which is an isometric view of the implantable medical lead 10 and a pulse generator 15 for connection thereto. The pulse generator 15 may be a pacemaker, defibrillator, ICD or neurostimulator. As indicated in FIG. 1, the pulse generator 15 includes a can 20, which houses the electrical components of the pulse generator 15, and a header 25. The header 25 is mounted on the can 20 and configured to receive a lead connector end 35 in a lead receiving receptacle 30.

As shown in FIG. 1, in one embodiment, the lead 10 includes a proximal end 40, a distal end 45 and a tubular body 50 extending between the proximal and distal ends. The proximal end 40 includes the lead connector end 35 including a pin contact 55, a first ring contact 60, a second ring contact 61, which is optional, and sets of spaced-apart radial seals 65. In some embodiments, the lead connector end 35 includes the same or different seals and may include a greater or lesser number of contacts. For example, the lead connector end 35 may be in the form of an IS-1, IS-4, DF-1, etc. configuration. In some embodiments, the lead connector end 35 includes the sacrificial welding tab 125 on one or more of the contacts 55, 60, 61. The lead connector end 35 is received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 1, in one embodiment, the lead distal end 45 includes a distal tip 70, a tip electrode 75 and a ring electrode 80. In some embodiments, the lead distal end 45 includes a helical anchor that is extendable from within the distal tip 70 for active fixation and may or may not act as an electrode. The helical anchor may be at least partly provided in a lumen of the header including the sacrificial welding tab 125 connected to an inner coil or other conductor or structure supported by the lead body 50. In other embodiments, the lead distal end 45 includes features or a configuration that facilitates passive fixation.

As shown in FIG. 1, in some embodiments, the distal end 45 includes a defibrillation coil 82 about the outer circumference of the lead body 50. The defibrillation coil 82 may be located proximal of the ring electrode 80.

As illustrated in FIG. 1, in one embodiment where the lead 10 is configured for passive fixation, the tip electrode 75 forms the distal tip 70 of the lead body 50. The ring electrode 80 extends about the outer circumference of the lead body 50, proximal of the distal tip 70. In other embodiments, a distal end 45 configured for passive fixation includes a greater or lesser number of electrodes 75, 80 in different or similar configurations.

In one embodiment where the lead 10 is configured for active fixation, an atraumatic tip forms the distal tip 70 of the lead body 50, and the helical anchor electrode is extendable/retractable relative to the distal tip 70 through an opening in the distal tip 70. The ring electrode 80 extends about the outer circumference of the lead body 50, proximal of the distal tip 70. In other embodiments, a distal end 45 configured for active fixation includes a greater or lesser number of electrodes in different or similar configurations.

In one embodiment, the tip electrode 75 or helical anchor electrode is in electrical communication with the pin contact 55 via a first electrical conductor and the ring electrode 80 is in electrical communication with the first ring contact 60 via a second electrical conductor. In some embodiments, the defibrillation coil 82 is in electrical communication with the second ring contact 61 via a third electrical conductor. In yet other embodiments, other lead components (e.g., additional ring electrodes, various types of sensors, etc.) mounted on the lead body distal region 45 or other locations on the lead body 50 are in electrical communication with a third ring contact (not shown) similar to the second ring contact 61 via a fourth electrical conductor. In some embodiments, one or more of the ring contacts may include the sacrificial welding tab 125 for electrically and mechanically coupling the ring contacts to the electrical conductors.

Depending on the embodiment, electrical connections in a lead body 50 between a location 110 on an electrical conductor 100 of the lead 10 and the electrical component or device 80 (e.g., an electrode for sensing or pacing, a defibrillation coil, a strain gage, a pressure sensor, an integrated chip, an inductor, a position tracking sensor, etc.) of the lead 10 served by the electrical conductor are accomplished via welding, crimping or a combination of welding and crimping. Crimp-thru technology employing thin-walled crimps or tubes 120 has several useful benefits including facilitating the manufacture of leads 10 having bodies 50 with minimized diameters and reducing manufacturing costs. Crimp-thru technology with thin-walled components 120 allows the thin-walled crimp 120 to be crimped directly through the electrical insulation (e.g., ETFE liner) jacketing the cable conductors 100, which removes a costly pre-ablation process on the cable conductors 100.

Current welding techniques have proven challenging when welding onto thin-walled crimp-thru crimps 120 because the elevated weld energy melts the thin metallic components causing weld penetration to the underlying ETFE insulation, which then vaporizes the ETFE and destroys the weld integrity. To address the issues presented by welding to a thin-walled crimp-through crimp 120, a component 80 (e.g., an electrode for sensing or pacing, a defibrillation coil, a strain gage, a pressure sensor, a piezoelectric sensor, an integrated chip, an inductor, a position tracking sensor, etc.) having a welding tab 125 has been developed and is described in detail below. The welding tab 125 of the component 80 allows for a lower energy weld due to the concentration of the energy on the sacrificial weld tab 125. This low energy weld does not penetrate down to the ETFE insulation and allows for consistent welding to a thin-walled crimp-thru crimp 120.

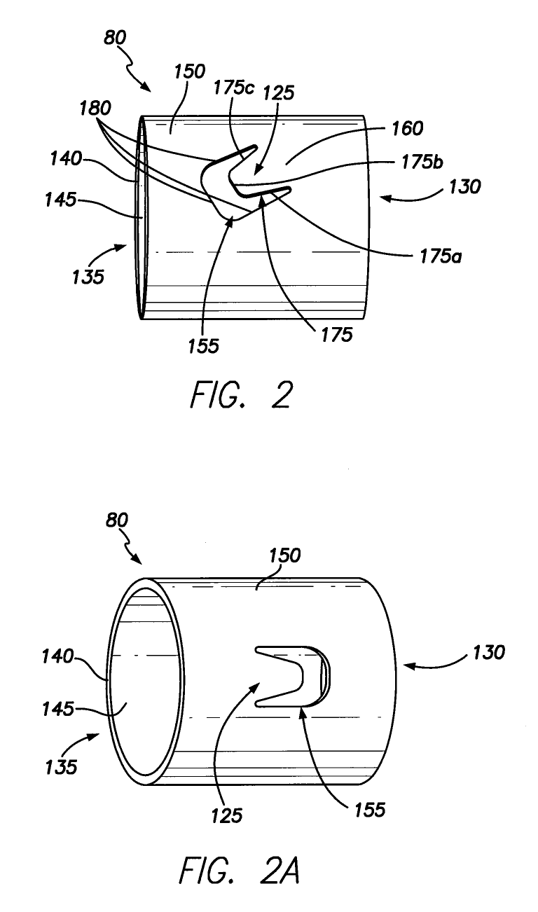

For a detailed discussion regarding a component 80, such as, for example, a ring electrode 80, employing the welding tab 125, reference is now made to FIG. 2, which is an isometric view of the ring electrode 80. As shown in FIG. 2, the ring electrode 80 is in the form of a thin-walled cylindrical body having an open circular distal end 130, an open circular proximal end 135, and a cylindrical wall 140 extending between the ends 130, 135 and defining an inner circumferential surface 145 and an outer circumferential surface 150.

As indicated in FIG. 2, the welding tab 125 may be considered to have a peninsula configuration. In other words, the welding tab 125 is defined in the cylindrical wall 140 so as to extend continuously and uninterrupted from the rest of the cylindrical wall 140 so as to project into a surrounding space or void 155 defined in and through the cylindrical wall 140. On account of the peninsular welding tab 125 projecting into the void 155, the welding tab 125 can be considered to include a side or region 160 that extends continuously and uninterrupted from the rest of the cylindrical wall 140 such that the tab inner surface and the tab outer surface run continuous and uninterrupted from the inner and outer circumferential surfaces 145, 150, respectively. Due to the peninsular welding tab 125 projecting into the void 155, the welding tab 125 can be considered to have a free border edge 175 that defines one side of the void 155 and may have multiple side segments 175a-c that define sides of the peninsular welding tab 125 that border the void 155.

As illustrated in FIG. 2, the void 155 is an opening defined in and through the cylindrical wall 140, thereby placing the inner and outer circumferential surfaces 145, 150 in communication with each other through the cylindrical wall 140. The boundaries of the void 155 are defined by the free border edge 175 of the tab 125 on one side and another edge 180 of the cylindrical wall 140 defined by, and across, the void 155 from the free border edge 175. The void 155 may have a horseshoe shape with the peninsular welding tab 125 located between the two side legs or extensions of the horseshoe shape.

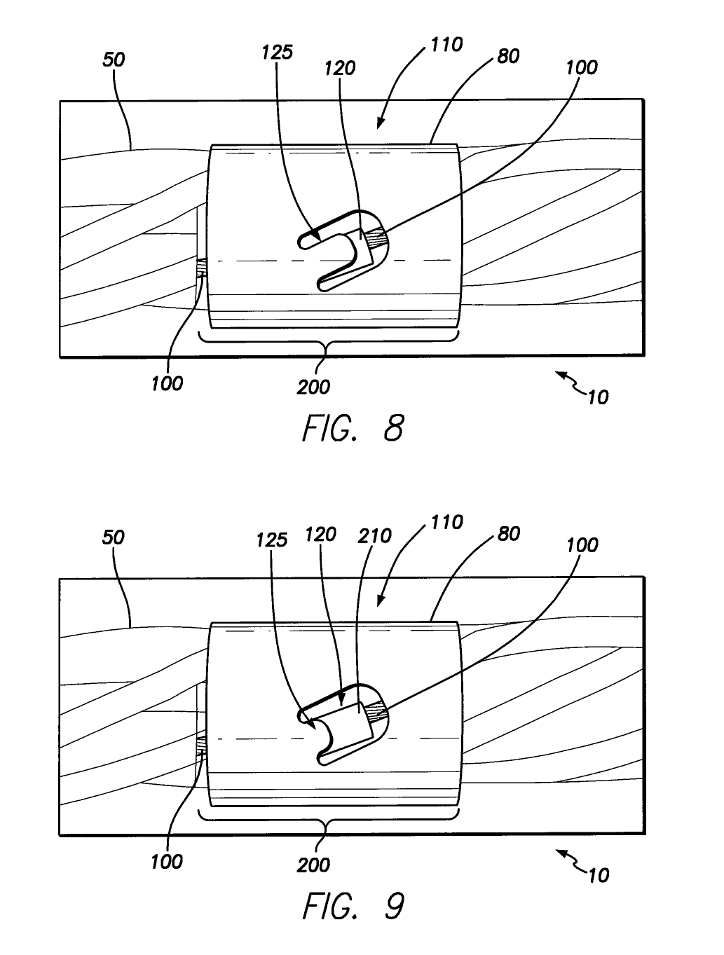

The welding tab 125 may be positioned be at any angle to match the orientation and shape of the underlying crimp 120. For example, as indicated in FIG. 2, in one embodiment, the welding tab 125 may be oriented such that its longitudinal axis is angled relative to the longitudinal distal-proximal axis of the ring electrode 80, thereby allowing the welding tab 125 to extend along the crimp 120, which is angled on account of being mounted on a conductor 100 helically extending through the lead body 50 (e.g., see FIGS. 7 and 8). In other embodiments, as illustrated in FIG. 2A, the welding tab 125 may be oriented such that its longitudinal axis is generally parallel to the distal-proximal longitudinal axis of the ring electrode.

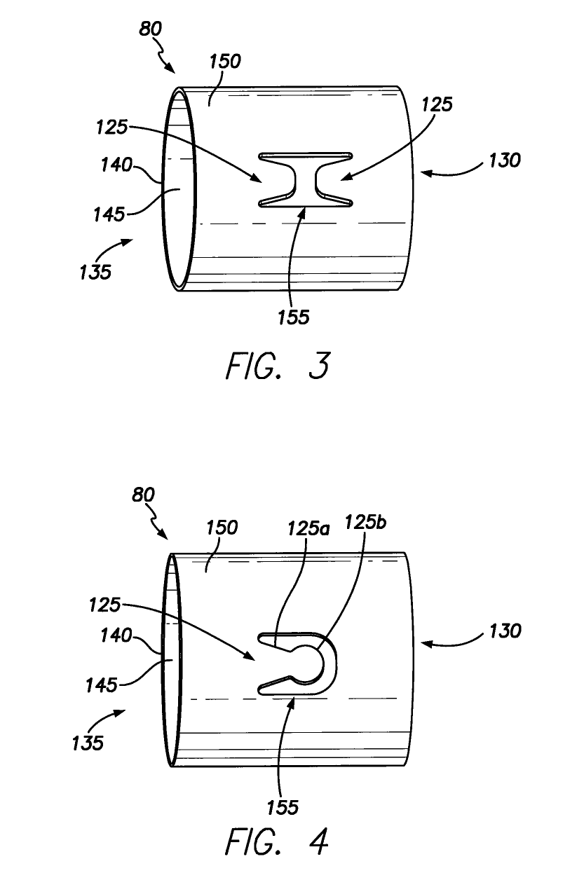

Depending on the embodiment, the tab-void configuration may be a single peninsular configuration with a horseshoe shaped void as discussed above with respect to FIG. 2 or may have a multiple peninsular configuration. For example, in one embodiment as shown in FIG. 3, which is the same view as FIG. 2, except of a multi-peninsular configuration, there may be two or more peninsular welding tabs 125 defined by a single void 155. In one embodiment, the there are two tabs 125, which are directly opposite each other across the void 155. The tabs 125 are configured similar to as described with respect to FIG. 2, and, since the tabs 125 project directly towards each other in an opposed fashion across the void 155, the void 155 can be said to have an H-shaped appearance. The multi-tabbed configuration of FIG. 3 may increase the mechanical strength of a weld to the crimp 120 formed via the multiple tabs 125.

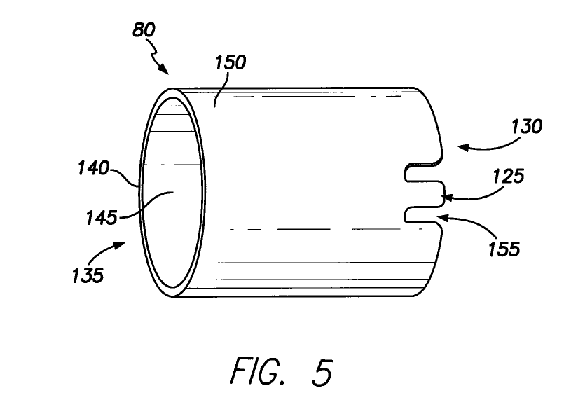

The opposed two-tab configuration of FIG. 3 is but one example of a multi-tab configuration. In another multi-tab configuration, one tab 125 may be positioned and configured similar to that depicted in FIG. 2, while the other tab 125 may be configured and located as indicated in FIG. 5 below. In other words, one tab 125 may be generally centered proximal-distal on the ring electrode 80 and the other tab 125 may be defined in one of the proximal or distal edges of the cylindrical wall 140 of the ring electrode 125.

The ring electrodes 80 of FIGS. 2 and 3 may be formed of a biocompatible metal such as, for example, platinum, platinum-iridium alloy, stainless steel, etc. The welding tab 125 can be manufactured into the ring electrode 80 via a variety of methods. For example, where the ring electrode 80 has sufficient thickness and size, the welding tab 125 may be machined into the ring electrode. Where the ring electrode 80 is too small or thin-walled for machining, manufacturing methods such as, for example, plunge/wire EDM or laser cutting technology may be employed to define the welding tab 125 in the ring electrode.

Laser technology is advantageous as it allows platinum parts to be cut into nearly any shape. As a result, laser technology may be used to define in the ring electrode 80 one or more welding tabs 125 of nearly any shape. For example, a peninsular welding tab 125 may have a shape that is different from the trapezoidal or truncated triangle shape depicted in FIG. 4. As depicted in FIG. 4, which is the same view as FIG. 2, except of a tab 125 having a different shape, in one embodiment, the peninsular welding tab 125 has a conical base 125a extending from the rest of the cylindrical wall 140, the conical base 125a transitioning into a circular-shaped free end 125b. Such a shaped welding tab 125 is tailored to take advantage of a circle weld spot. Further, such a shaped welding tab 125 provides a benefit for the operator who can easily target the laser welding beam on the center of the circular-shaped free end 125b of the welding tab 125. Of course, such a shaped welding tab 125 is merely an example of the numerous configurations a welding tab 125 may take.

Depending on how the overall component 80 is to appear in its finished state, the defining of the welding tab 125 may occur at different points in the manufacturing of the component. For example, where the component 80 is a ring electrode 80 or other similar cylindrical, thin-wall component, the ring electrode 80 may be stamped, rolled, and welded shut at the seam. The welding tab 125 could be defined in the ring electrode 80 prior to being rolled or after the ring electrode is welded shut.

Rather than being positioned in the center of the ring electrode 80 as depicted in FIG. 2, the welding tab 125 may be located in a variety of other locations on the ring electrode 80. For example, as illustrated in FIG. 5, which is the same view as FIG. 2, except of an alternative welding tab location, the welding tab 125 can be defined in edge of the cylindrical wall 140 of one of the distal or proximal ends 130. The welding tab 125 can still be seen to have a peninsular shape. FIG. 5 also illustrates a welding tab 125 of yet another shape, which is generally rectangular with rounded corners, although the welding tab 125 may employ the above-described trapezoidal shape or other shapes.

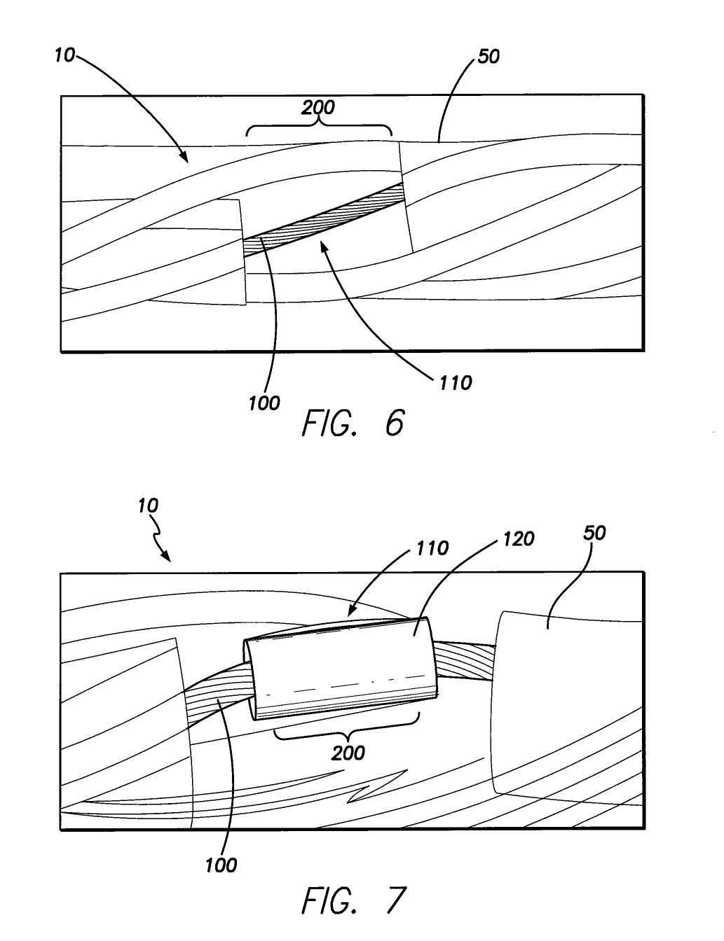

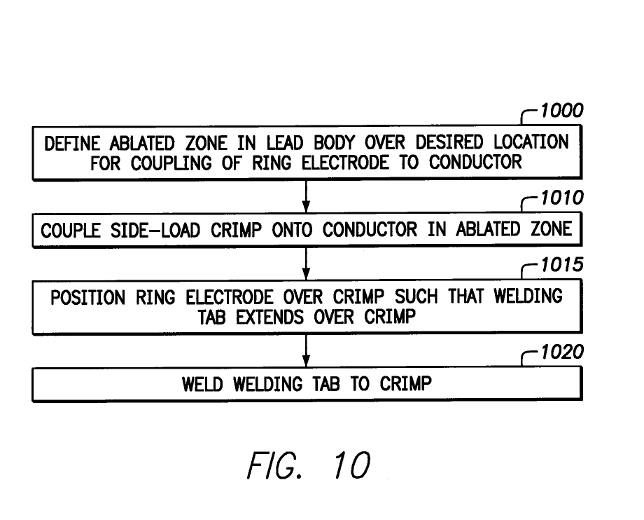

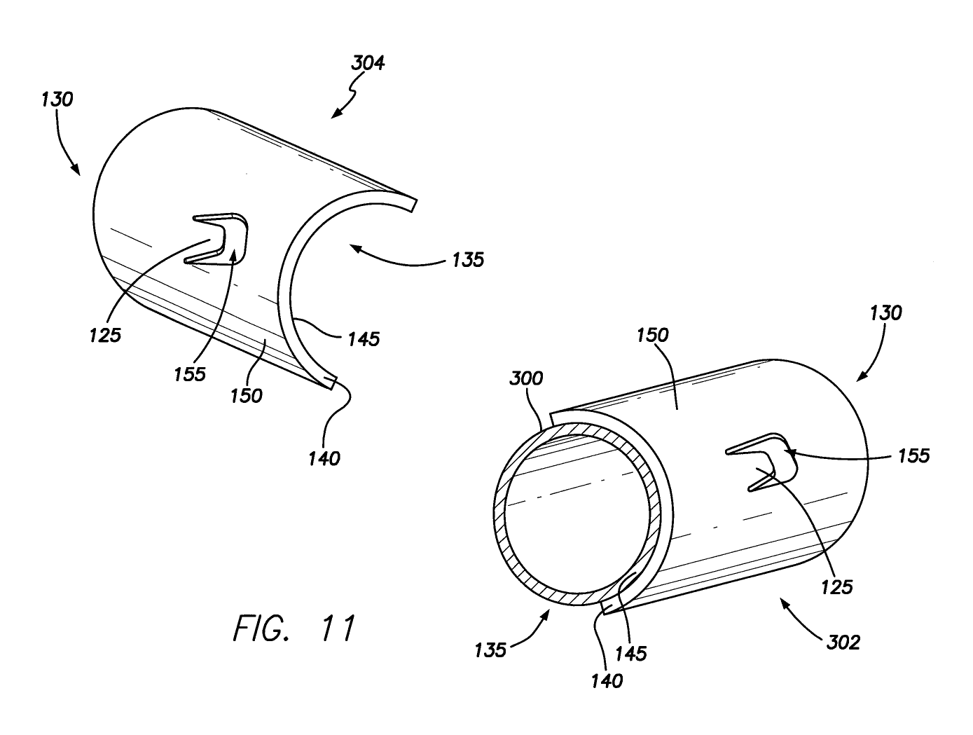

For a discussion of a manufacturing method used to electrically and mechanically couple the ring electrode 80 to an electrical conductor 100 extending through the lead body 50, reference is made to FIGS. 6-10. FIGS. 6-9 are successive plan views of the ring electrode 80 being mounted on the lead body 50 and electrically and mechanically coupled to the conductor 100. FIG. 10 is a flow chart outlining the method.

As shown in FIG. 6, an ablated zone 200 is defined in the lead body at the location 110 of the electrical and mechanical coupling of the ring electrode 80 to the conductor 100 [block 1000 of FIG. 10]. Specifically, the ablated zone extends through the polymer layers of the lead body 50 and the insulation jacket of the electrical cable conductor 100 to which the ring electrode 80 is to be electrically and mechanically coupled. The ablated zone 200 provides access to the electrical conductor 100 to allow for a crimp 120 to be side-loaded onto and crimped down onto the electrical conductor 100.

As illustrated in FIG. 7, a side-load crimp 120 is mechanically coupled at the location 110 to the exposed electrical conductor 100 [block 1010 of FIG. 10]. In one embodiment, the crimp 120 may be a tube 120 or other structure that is crimped, welded or otherwise mechanically and electrically coupled to the electrical conductor 100 at the location 110. In being so coupled to the electrical conductor 100 at the location 110, the crimp or tube 120 may extend about at least a portion of an outer circumference of the electrical conductor 100 at the location 110.

As can be seen in FIG. 7, because of the spiral routing of the conductor 100 through the lead body 50 and the cylindrical configuration of the crimp 120, the crimp 120 ends up being oriented at an angle relative to the longitudinal axis of the lead body 50 and, therefore, oriented at an angle relative to the inner circumferential surface of the ring electrode 80. This misalignment between the crimp 120 and electrode 80 can make the side-load crimp 120 more challenging to weld to the ring electrode 80 as compared to traditional crimps that are crescent shaped and match the inner diameter of the ring electrode because the surface contact or points between the ring electrode and the side-load crimp are reduced by the misalignment. As can be understood from FIGS. 8 and 9, the welding tab 125 arrangement of the ring electrodes 80 disclosed herein help to ease the welding difficulty associated with the misalignment between the ring electrode and the crimp.

As depicted in FIG. 8, the ring electrode 80 is positioned on the lead body 50 such that the welding tab 125 is positioned over the side-load crimp 120 [block 1015 of FIG. 10]. The orientation of the welding tab 125 relative to the rest of the ring electrode 80 may be such that the weld tab 125 extends along the side-load crimp 120.

As illustrated in FIG. 9, the welding tab 125 is welded to the side-load crimp 120 [block 1020 of FIG. 10]. The resulting weld is robust, and the polymer layers of the lead body 50 and the electrical insulation jacket of the conductor 100 have not been adversely impacted by the welding process. The configuration of the welding tab 125 results in weld nugget 210 that is thicker and stronger than would otherwise be possible with such low welding energy as employed in making the weld nugget 210.

As can be understood from FIG. 2, the welding tab 125 is part of the cylindrical wall 140 of the ring electrode 80 via one tab side or region 160 being an extension of the cylindrical wall 140. However, the other three tab sides 175a-c are isolated from the rest of the cylindrical wall 140. As a result, the welding tab 125 can be used as an isolated, sacrificial welding tab 125 offering certain benefits. For example, as can be understood from FIGS. 8 and 9, the isolated, sacrificial welding tab 125 defined in the wall 140 of the ring electrode 80 allows for concentration of the heat from welding in a localized area. As a result, more molten metal is generated for fusion between the ring electrode 80 and the underlying crimp 120. Specifically, during welding, the laser energy melts the welding tab 125, thereby generating a relatively large welding pool which then fills the gap between the components (i.e., the ring electrode 80 and the crimp 120) and fuses them together. The use of the isolated, sacrificial welding tab 125 also allows for a low energy weld with less welding heat propagating into the lead body 50 and underlying crimp 120 and conductor 100.

For a detailed discussion regarding the lead 10 having a plurality of electrical components 80, each employing a welding tab 125, reference is now made to FIG. 11, which is an isometric view of a split ring electrode, showing a first electrical component 302 extending about a circumference of electrical insulation 300 (e.g., ETFE liner) and a second electrical component 304 separate from the insulation 300. In one embodiment, the split ring electrode is mounted on the lead body 50 such that the first electrical component 302 is arranged generally opposite the second electrical component 304.

As indicated in FIG. 11, the first and second electrical components 302, 304 are each in the form of a portion (e.g., approximately half) of a thin-walled cylindrical body having an open circular distal end 130, an open circular proximal end 135, and a cylindrical wall 140 extending between the ends 130, 135 and defining an inner circumferential surface 145 and an outer circumferential surface 150. In some embodiments, the first and second electrical components 302, 304 are electrically isolated from each other when mounted on the lead body 50, thereby forming independent electrode surfaces.

The welding tabs 125 on each of the first and second electrical components 302, 304 may be considered to have a peninsula configuration. In other words, the welding tab 125 is defined in the cylindrical wall 140 of each of the first and second electrical components 302, 304 so as to extend continuously and uninterrupted from the rest of the cylindrical wall 140 so as to project into a surrounding space or void 155 defined in and through the cylindrical wall 140.

As illustrated in FIG. 11, the voids 155 are an opening defined in and through the cylindrical walls 140 of the first and second electrical components 302, 304, thereby placing the inner and outer circumferential surfaces 145, 150 in communication with each other through the cylindrical walls 140. The voids 155 may have a horseshoe shape with the peninsular welding tabs 125 located between the two side legs or extensions of the horseshoe shape. Depending on the embodiment, the tab-void configuration may be a various other configurations as discussed herein.

The welding tabs 125 may be positioned be at any angle to match the orientation and shape of underlying crimps 120. For example, as indicated in FIG. 11, in one embodiment, the welding tabs 125 of the first electrical component 302 may be oriented such that its longitudinal axis is angled relative to the longitudinal distal-proximal axis, thereby allowing the welding tab 125 to extend along the crimp 120, which is angled on account of being mounted on a conductor 100 helically extending through the lead body 50. In other embodiments, as illustrated herein, the welding tab 125 may have other orientations.

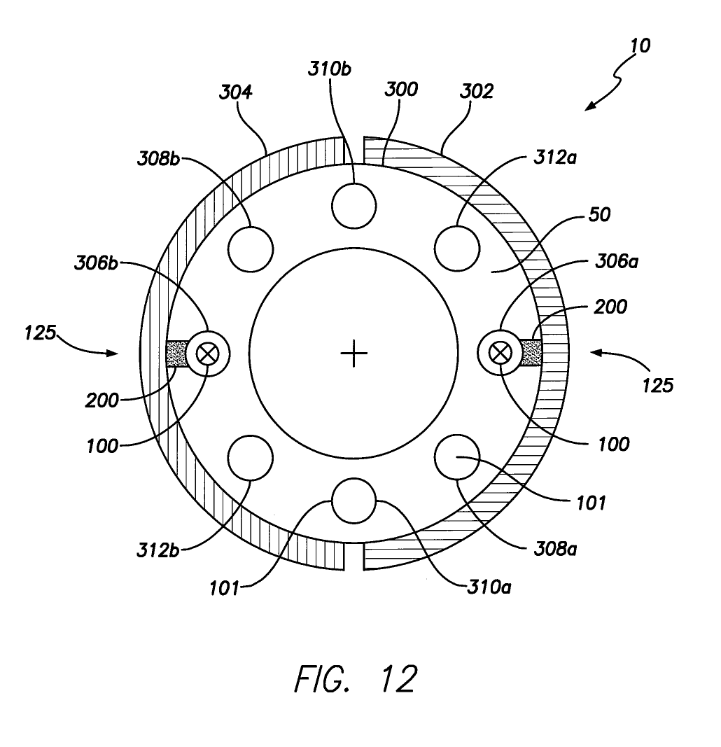

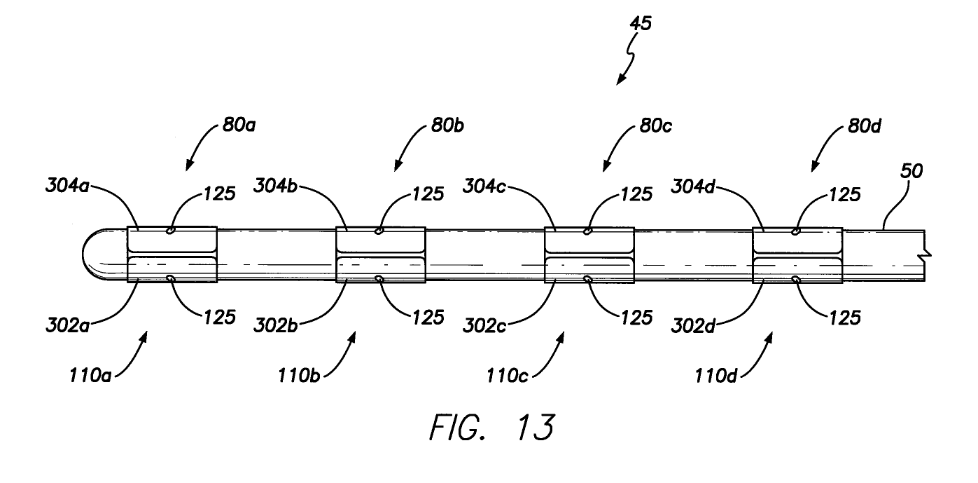

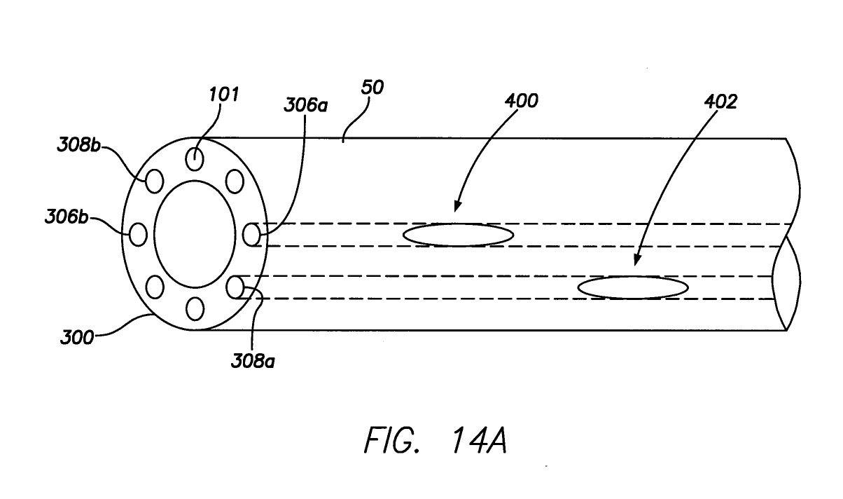

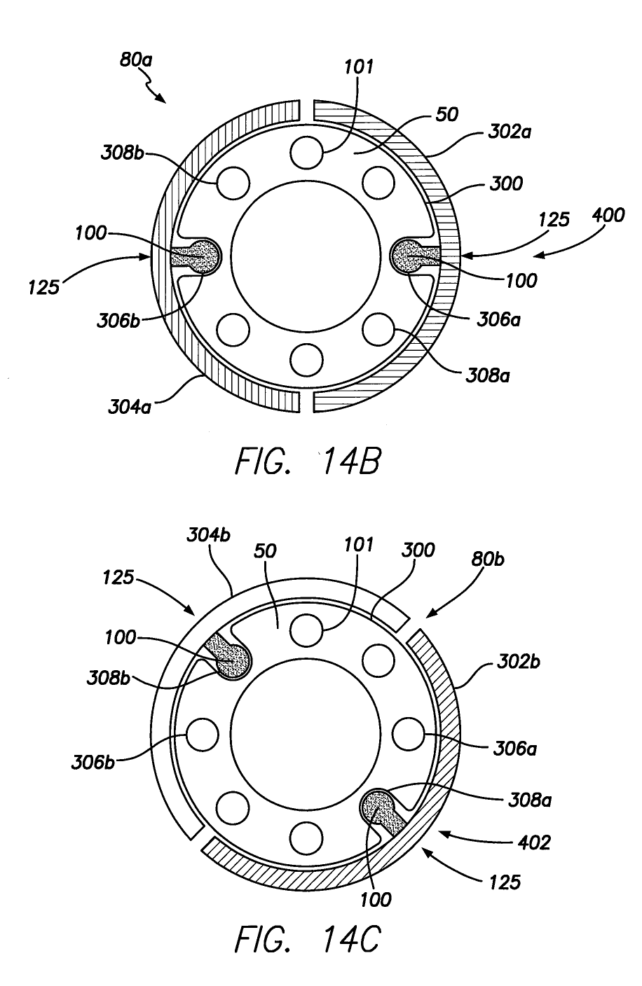

As can be understood from FIG. 12, which is a cross-sectional view of the lead 10, in some embodiments, a plurality of electrical conductors 100 extend through the lead body 50 via lumens 101. The lumens 101 and the electrical conductors 100 therein may be arranged in a rotary pattern. As shown in FIG. 12, the conductors are arranged such that a plurality of split ring electrodes may be mounted on the lead body 50 such that the independent electrical surfaces of each split ring electrode are positioned generally opposite. For example, when the first electrical component 302 of a first split ring electrode is electrically and mechanically connected to a first electrical conductor 306a and the second electrical component 304 is electrically and mechanically connected to a second electrical conductor 306b, the first and second electrical components 302, 304 are arranged generally opposite each other. Similarly, as can be understood from FIGS. 13-14C, electrical components may be electrically and mechanically coupled to additional electrical conductors 100 (e.g., the electrical conductors 308a, 308b, 310a, 310b, 312a, and 312b) routed through the wall lumens 101 to create an independent electrical surface corresponding to each conductor.

As described herein, an electrical connection between the electrical conductor 306a and the electrical component 302 or between the electrical conductor 306b and the electrical component 304 may be accomplished via welding, crimping or a combination of welding and crimping. Crimp-thru technology with thin-walled components 120 allows the thin-walled crimp 120 to be crimped directly through the electrical insulation 300. As discussed above with respect to FIGS. 6-9, for each location 110 of the electrical and mechanical coupling, an ablated zone 200 extends through the polymer layers of the lead body 50 and the insulation 300. The ablated zones 200 provide access to the electrical conductors 306a, 306b to allow for a crimp 120 to be side-loaded onto and crimped down onto the electrical conductors 306a, 306b. The welding tabs 125 of the first and second electrical components 302, 304 may be positioned be at any angle to match the orientation and shape of underlying crimps 120.

Turning to FIG. 13, which is a plan view of the lead distal end 45, a plurality of split ring electrodes are mounted on the lead body 50 and electrically and mechanically coupled to a plurality of conductors. In the embodiment shown in FIG. 13, the electrical components are arranged in pairs at a plurality of locations 110. Specifically, first and second electrical components 302a, 304a of a first split ring electrode 80a are mounted at a first location 110a; first and second electrical components 302b, 304b of a second split ring electrode 80b are mounted at a second location 110b; first and second electrical components 302c, 304c of a third split ring electrode 80c are mounted at a third location 110c; and first and second electrical components 302d, 304d of a fourth split ring electrode 80d are mounted at a fourth location 110d. Each of the electrical components is electrically and mechanically coupled to a respective conductor via a welding tab 125. The configuration shown in FIG. 13 provides eight independent conducting surfaces on the lead distal end 45.

FIG. 14A is a perspective cross-sectional view of a first location 400 where the first electrical component 302a of a first split ring electrode 80a is electrically and mechanically coupled to the electrical conductor 306a and a second location 402 where the first electrical component 302b of the second split ring electrode 80b is electrically and mechanically coupled to the electrical conductor 308a. FIGS. 14B-14C are cross-sectional views of the first location 400 and the second location 402, respectively. As can be understood from FIGS. 14A-14C, the split ring electrodes may be mounted on the lead body 50 in pairs of electrical components arranged about the outer circumference of the lead body 50 in locations corresponding to the respective electrical conductor to which the electrical component is to be electrically and mechanically coupled.

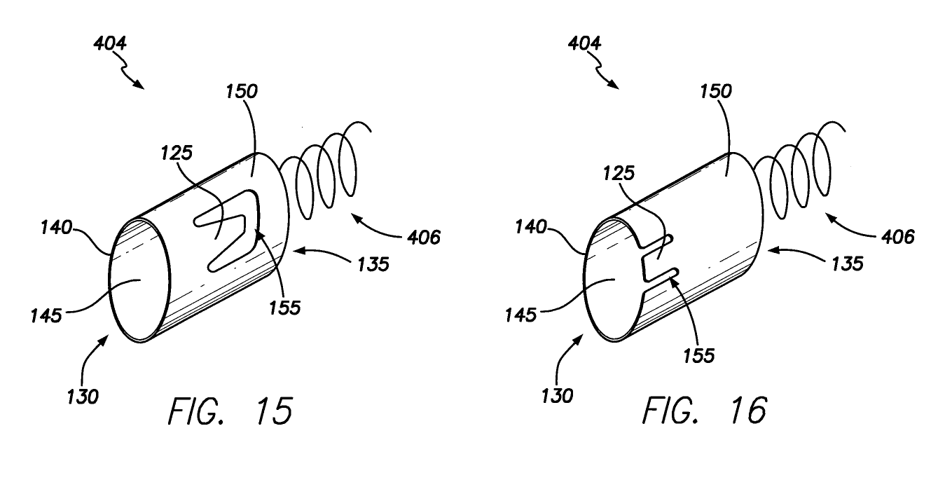

For a detailed discussion of a header 404 employing the welding tab 125, reference is made to FIGS. 15 and 16. A helical anchor 406 that is extendable from within the distal tip 70 of the lead 10 for active fixation and may or may not act as an electrode. The helical anchor 406 may be at least partly provided in a lumen of the header 404. In some embodiments, the sacrificial welding tab 125 is connected to an inner coil or other conductor. In other embodiments, the sacrificial welding tab 125 is connected to a structure supported by the lead body 50.

As can be understood from FIG. 16, rather than being positioned in the center of the header 404 as depicted in FIG. 15, the welding tab 125 may be located in a variety of other locations on the header 404. For example, as illustrated in FIG. 16, which is the same view as FIG. 15, except of an alternative welding tab location, the welding tab 125 can be defined in edge of the cylindrical wall 140 of one of the distal or proximal ends 130 of the header 404. The welding tab 125 can still be seen to have a peninsular shape. Other shapes and locations are also contemplated as described herein.

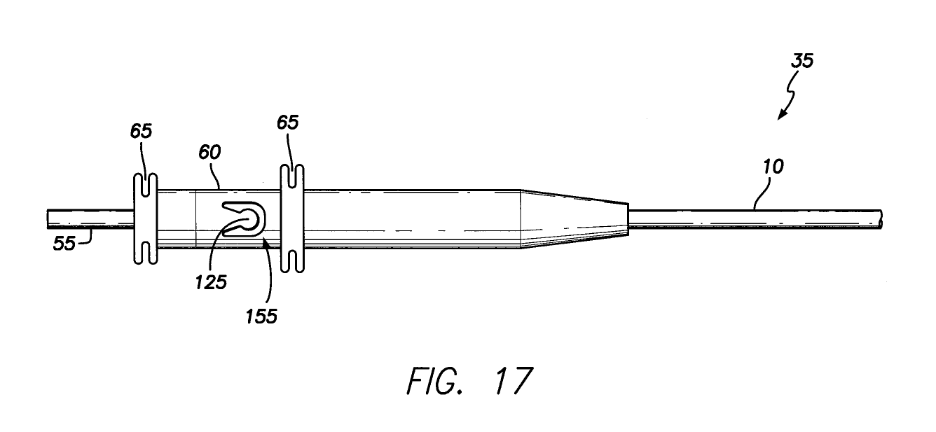

FIG. 17 is a plan view of the lead connector end 35 employing the welding tab 125. In one embodiment, the lead connector end 35 includes the pin contact 55, a ring contact 60 including the welding tab 125, and sets of spaced-apart radial seals 65. In some embodiments, the lead connector end 35 includes the same or different seals and may include a greater or lesser number of ring contacts, each of which may include the welding tab 125. As described herein, the lead connector end 35 may be in the form of an IS-1, IS-4, DF-1, etc. configuration. In the embodiment shown in FIG. 17, the lead conductor end 35 is in the form of an IS1 configuration.

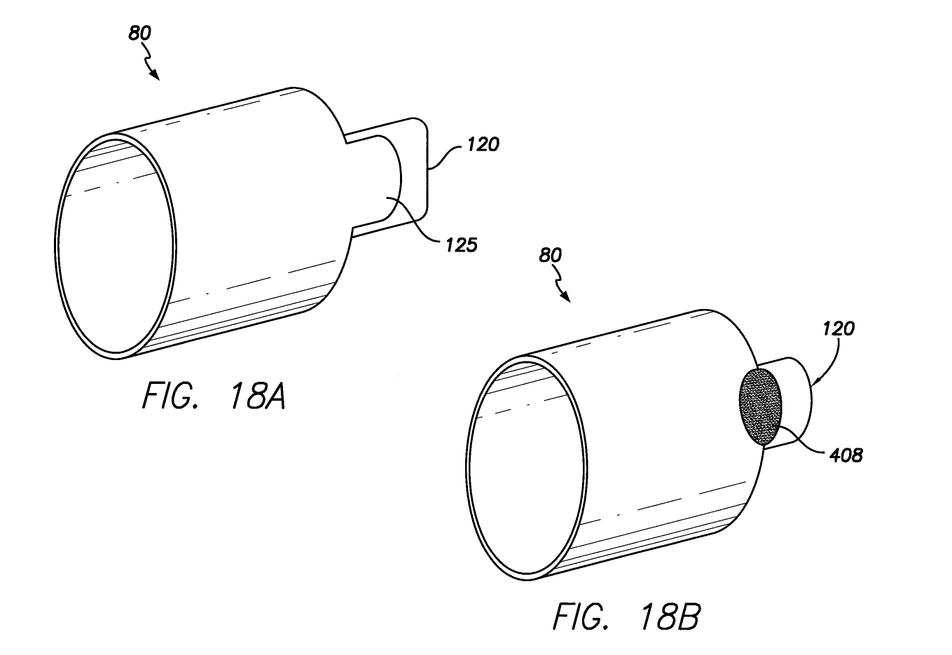

FIGS. 18A-18B illustrated the extended welding tab 125 before and after welding, respectively. As shown, once the welding tab 125 is welded to the crimp or tube 120 to form a weld 408, for example, a smooth, seamless transition from the electrical component 80 to the crimp or tube 120 is created.

While the above-described embodiments are given in the context of the component 80 being a ring electrode 80, a split ring electrode, a header 404, or a lead connector end 35, it should be noted that the above-described welding tab configurations and associated teachings may be applied to other components 80 including, for example, shock coils or other components that weld in a similar fashion to the electrical components described herein. The welding tab configurations and associated teachings disclosed herein may also apply for other termination methods such as, for example, making electromechanical connections to sensors.

Although the present invention has been described with reference to preferred embodiments, persons skilled in the art will recognize that changes may be made in form and detail without departing from the spirit and scope of the invention.

What is claimed is:

1. A method of assembling an implantable medical lead, the method comprising:
supporting an electrical component on a lead body, the electrical component comprising a sacrificial feature defined in a wall of the electrical component, the sacrificial feature comprising a region that continues from the wall of the electrical component and a side that is isolated from the wall of the electrical component via a void defined in the wall of the electrical component; and
welding at least a portion of the sacrificial feature, a resulting weld operably coupling the electrical component to an electrical conductor extending through the lead body.

2. The method of claim 1, wherein the electrical component comprises a ring electrode or a defibrillation coil.

3. The method of claim 1, wherein the electrical component comprises a strain gage, a pressure sensor, a piezoelectric sensor, an integrated chip, an inductor, or a position tracking sensor.

4. The method of claim 1, further comprising securing a crimp to the electrical conductor, and also welding at least a portion of the crimp to form at least a part of the resulting weld.

5. The method of claim 4, wherein the crimp is secured to the electrical conductor via a crimp-thru type crimping process.

6. The method of claim 1, wherein the sacrificial feature comprises a welding tab.

7. The method of claim 6, wherein, prior the welding the at least a portion of the sacrificial feature, the welding tab is peninsular within the void defined in the wall of the electrical component.

8. The method of claim 1, wherein the wall of the electrical component comprises a distal end edge and a proximal end edge, and the void is defined in the wall between, and spaced away from, the distal end edge and the proximal end edge.

9. The method of claim 1, wherein the wall of the electrical component comprises a distal end edge and a proximal end edge, and the void is defined in the wall at either the distal end edge or the proximal end edge.

10. The method of claim 1, wherein, prior the welding the at least a portion of the sacrificial feature, a longitudinal axis of the sacrificial feature extends along a longitudinal axis of the electrical conductor at the weld.

11. The method of claim 1, wherein, prior the welding the at least a portion of the sacrificial feature, the sacrificial feature comprises a peninsular shape within the void, the peninsular shape comprising a trapezoidal shape, a rounded rectangular shape, or a conical shape terminating in a circular free end.
